SEQUENCE LISTING (12 sequences)

SEQ ID NO: 1 - DNA, 24 bp, Artificial
  primer.molecular weight =7920Da
ttgatatcatgtcgacctatccag

SEQ ID NO: 2 - DNA, 24 bp, Artificial
  primer.molecular weight =7920Da
ttcgttccgtcctgcagcctcaat

SEQ ID NO: 3 - DNA, 18 bp, Artificial
  primer.molecular weight = 5940Da
gcaagcttggcgattaca

SEQ ID NO: 4 - DNA, 21 bp, Artificial
  primer.molecular weight = 6930Da
tgccaggatatcagacagatg

SEQ ID NO: 5 - DNA, 13 bp, Artificial
  primer.molecular weight = 4290Da
ggccaacgcggcc

SEQ ID NO: 6 - DNA, 11 bp, Artificial
  primer.molecular weight = 3630Da
cctgcagcaga

SEQ ID NO: 7 - DNA, 10 bp, Artificial
  RAPD primer 1
ggtgcgggaa

SEQ ID NO: 8 - DNA, 10 bp, Artificial
  RAPD primer 2
gtttcgctcc

SEQ ID NO: 9 - DNA, 10 bp, Artificial
  RAPD primer -3
gtagacccgt

SEQ ID NO: 10 - DNA, 10 bp, Artificial
  RAPD primer 4
aagagcccgt

SEQ ID NO: 11 - DNA, 10 bp, Artificial
  RAPD primer 5
aacgcgcaac

SEQ ID NO: 12 - DNA, 10 bp, Artificial
  RAPD primer 6
cccgtcagca

(19) United States
(12) Patent Application Publication
(10) Pub. No.: US 2011/0086343 A1
(43) Pub. Date: Apr. 14, 2011

(54) METHOD FOR THE SCREENING OF BACTERIAL ISOLATES

(76) Inventors: Hemant Jyotiswarup Purohit, Maharashtra (IN); Atya Kapley, Maharashtra (IN); Dhananjay Vasant Raje, Maharashtra (IN); Sukumar Devotta, Maharashtra (IN)

(21) Appl. No.: 12/282,376

(22) PCT Filed: Jan. 22, 2007
(86) PCT No.: PCT/IB07/00136

§ 371 Date: Dec. 8, 2008

(30) Foreign Application Priority Data
Mar. 10, 2006 (IN) 0651/DEL/2006

(51) Int. Cl. C12Q 1/68

(52) U.S. Cl. 435/6

(57) ABSTRACT

Present invention relates to a method to determine the genotype of organisms by RAPD analysis and more specifically, to establish the relatedness of individual organisms across and within species. RAPD uses genotypic information of an organism to give an organism specific DNA fragment of different sizes. The present invention provide methods and a set of oligonucleotide primers for performing amplification and other enzymatic reactions on nucleic acid molecules that have been collected directly as environmental DNA or DNA derived form pure isolates. More specifically, the present invention relates to a novel method of genetic analysis using a set of sub-sequence, which occurs as inverted repeats in different genome with different frequencies. All bacterial cultures used in this study have been isolated from activated biomass collected from effluent treatment plants. The bacteria have been sub-cultured repeatedly to obtain pure cultures. All plating has been carried out on Luria Broth plates with 2% agar. The 16S rRNA gene has been amplified using universal primers to confirm the eubacterial nature of the isolates. The primers used to amplify a 1466-bp product were 27F forward primer 5′-AGAGTTTGATCMTGGCTCAG-3′ and 1492 reverse primer 5′-TACGGYTAC-CTTGTTACGACTT-Hence, in defined conditions two genome samples could be differentiated from each other. These features are applicable to DNA fingerprinting, marker assisted selection, genotyping, and high throughput laboratory screening methods for culturable microbes from any environmental niche.

### FIELD OF INVENTION

The present invention relates to a composition of primers useful for the screening of bacterial isolates.

The present invention also relates to a method for the screening of bacterial isolates. Further, it also relates to a kit for the screening of bacterial isolates.

### BACKGROUND AND PRIOR ART OF INVENTION

One of the most important tasks in microbial ecology and its application in bioremediation technology is the precise determination of microorganisms in the environmental samples (Chhatre et al. 1996, Fritsche and Ofrichter, 1999; Dubey et al. 2003). Using some commonly known and simple techniques, the microbiologist can quickly deduce the species of the unknown microorganism. However, subspecies or actual strain determination of the microorganism present in the sample frequently requires sophisticated methods of genetic or biochemical analysis (Amman et al., 1995; Gich et al. 2000). This becomes particularly more relevant, if few selected genera are over-represented and are contributing for most of the available physiological diversity (Benes et al. 2002; du Plessis, 2001; Kapley et al. 2001; Mahendraker, and Viraraghavan, 2001; Orupold 2001; Rittman, 2002; Saadoun, 1999). The analysis of this kind generally translates into higher costs and longer durations.

The microbial population and its survival depends on the environmental conditions wherein a series of organic molecules acts as carbon source to support balance growth kinetics for the evolving microbial community (Atuanya et al. 2000; Qureshi et al. 2001, Qureshi and Purohit 2002; Padmanabhan et al. 2003; Purohit et al. 2003; Watanabe, 2002; Watanabe et al. 2002). At molecular level various gene pools contribute the desired metabolic information, which are provided by different genera; or even some time by same genera with different species having diverse catabolic capacity. At molecular level each such individual microbe can be described for its genotype. Genotypes can be more readily described in terms of genetic markers. A genetic marker identifies a specific region or locus in the genome (Vaneechhoutte, 1995). Thus, the more the genetic markers, the finer are the genotype. A genetic marker becomes particularly useful when it is allelic between organisms because it then may serve to unambiguously identify an individual. There are methods like Random Amplified Polymorphic DNA (RAPD), which have been reported for such analysis (Narde et al. 2004).

Reference may be made to Ikeda et al. (U.S. Pat. No. 5,665,572) wherein a method of amplifying template DNA by polymerase chain reaction (PCR) in which a single oligonucleotide primer having a restriction site is contacted with the template DNA. The oligonucleotide randomly anneals to a single strand of the template DNA and DNA sequences complementary to the single strand are synthesized. Furthermore reference may be made to Benson (U.S. Pat. No. 6,284,466) in which a method for the detection of polymorphisms has been disclosed on the basis of nucleotide differences in DNA sequence DNA sequences from different sources are amplified by PCR using primers based on strand bias, over represented oligonucleotide sequences such that the differences in the nucleotide sequences of the amplification products could be determined which is reported by Werker et al. (2003). Another reference may be made to Hall (U.S. Pat. No. 6,046,390), which relates to the inbred and hybrid genetic complements of the inbred corn plant 01INL1, and also to the RFLP and genetic isozyme typing profiles of inbred corn plant 01INL1. Reference may be made to Ozias-Akins et al. (U.S. Pat. No. 6,028,185), which relates to nucleic acid markers for an apospory-specific genomic region from the genus Pennisetum. Reference may be made to McClelland et al. (U.S. Pat. No. 5,487,985) in which a method based on the arbitrarily primed polymerase chain reaction (AP-PCR) has been described. This method is suitable for the identification of bacterial species and strains, including Staphylococcus and Streptococcus species, mammals and plants. Similarly, reference may be made to Kilian A. (U.S. Pat. No. 6,713,258) which disclose the method for determination of genotype of organisms by hybridization analysis to establish the relatedness of individual organisms within a species. Also reference may be made to Macevicz S. C. (U.S. Pat. No. 5,002,867) for providing a method for sequencing nucleic acids without the need to separate similarly sized DNAs or RNAs by gel electrophoresis. The method relies on the separate hybridization of multiple mixed oligonucleotide probes to a target sequence.

Amongst the various methods, RAPD provides the coverage of the genome in a single experiment where designed primer in the PCR reaction finds its immediate inverted repeats as a partner under the defined thermo cycling conditions. It allows the exponential amplification of DNA sub-string within the selected inverted repeat. Thereby, it works as a strong tool for discrimination of ancestrally related or even the variants for the same species. Earlier reports of random amplification using single primer describe only methods used to amplify trace amounts of target DNA, detection of polymorphisms in a DNA sequence and nucleic acids markers. However, there are no reports of methods that use designed oligonucleotide for fingerprinting DNA samples for distinguishing microbial diversity using RAPD.

### OBJECTS OF THE PRESENT INVENTION

The main object of the present invention is to provide a composition comprises of six primers useful for the screening of bacterial isolates.

Another object of the present invention is to provide a method useful for the screening of bacterial isolates.

Further another object of the present invention is to provide a sensitive test for diagnosis of the genetic diversity based on RAPD DNA pattern profile a kit useful for the screening of bacterial isolates.

### SUMMARY OF INVENTION

The present invention deals with a composition, which comprises of six primers useful for the screening of bacterial isolates. Further, the invention also relates to a method useful for screening of bacterial isolates based on nucleic acid sub-sequence, which are flanked by inverted repeats. It is particularly focus on sets of primers that are specifically evaluated for discriminating bacteria based on genotypic characteristics. The detection method employs a polymerase chain reaction (PCR) technique, using specific oligonucleotide primers for amplification. Further, it also provides a kit useful for the screening of bacterial isolates.

### BRIEF DESCRIPTION OF FIGURES

FIG. 1 represents RAPD of single template with different commercially available enzymes.

FIG. 2 represents RAPD with commercially available primers.

FIG. 3A-3B represents evaluation of suitable primers using genomic DNA isolated from bacteria present in Activated sludge.

FIG. 4A-4B and FIG. 5A-5C represents evaluation of all 31 primers using genomic DNA isolated from two different laboratory isolates.

FIG. 6A-6E represents performance of six selected primers at varying annealing temperatures.

FIG. 7A-7C represents performance of six primers at varying magnesium ion concentration.

FIG. 8-14 represents the PCR products at annealing temperature of 45° C.

FIG. 15-20 represents the PCR products using annealing temperature of 50° C.

FIG. 21-26 represents the PCR products at 45° C. and 50° C. temperature.

FIG. 27-32 represents the hairpin structure of the six primers derived using the Laser gene software DNASTAR, USA.

### DETAILED DESCRIPTION OF INVENTION

Accordingly, the present invention provides a composition comprising six primers useful for screening of bacterial isolates, wherein the primer sequences consisting of all or part of the following sequence ID;

a) SEQ ID NO 1: 5′-TTGATATCATGTCGACCTATCGAG-3′;
b) SEQ ID NO 2: 5′-TTCGTTCCGTCCTGCAGCCTCAAT-3′;
c) SEQ ID NO 3: 5′-GCAAGCTTGGCGATTACA-3′;
d) SEQ ID NO 4: 5′-TGCCAGGATATCAGACAGATG-3′;
e) SEQ ID NO 5: 5′-GGCCAACGCGGCC-3′;
f) SEQ ID NO 6: 5′-CCTGCAGCAGA-3′.

In an embodiment of the present invention, the said individual primers have the following characteristics:
a) SEQ ID NO 1 have the following characteristics:
   i. melting temperature (degree C.)=52.6 degree C.;
   ii. number of hairpin loops <2;
   iii. number of dimmers <2;
   iv. number of internal loops=2;
   v. molecular weight=7920 Da;
c) SEQ ID NO 2 have the following characteristics:
   i. melting temperature (degree C.)=64.5 degree C.;
   ii. number of hairpin loops <2;
   iii. number of dimmers <2;
   iv. number of internal loops=2;
   v. molecular weight=7920 Da;
d) SEQ ID NO 3 have the following characteristics:
   i. melting temperature (degree C.)=49.6 degree C.;
   ii. number of hairpin loops <2;
   iii. number of dimmers <2;
   iv. number of internal loops=2;
   v. molecular weight=5940 Da;
e) SEQ ID NO 4 have the following characteristics:
   i. melting temperature (degree C.)=49.9 degree C.;
   ii. number of hairpin loops <2;
   iii. number of dimmers <2;
   iv. number of internal loops=2;
   v. molecular weight=6930 Da
f) SEQ ID NO 5 have the following characteristics:
   i. melting temperature (degree C.)=51.5 degree C.;
   ii. number of hairpin loops <2;
   iii. number of dimmers <2;
   iv. number of internal loops=2;
   v. molecular weight=4290 Da
g) SEQ ID NO 6 have the following characteristics:
   i. melting temperature (degree C.)=59.5 degree C.;
   ii. number of hairpin loops <2;
   iii. number of dimmers <2;
   iv. number of internal loops=2;
   v. molecular weight=3630 Da

In another embodiment of the present invention, the said primers specifically hybridize to DNA inverted repeats and uniquely map different sites in total DNA of an environmental origin or eubacteria.

Further, in another embodiment of the present invention, the said primers are used for RAPD analysis for assessment of diversity of genera, different isolates or total DNA of any environmental or any origin.

In yet another embodiment of the present invention, the said primers help in high through put screening of bacterial isolates.

Further, the present invention also provides a method for screening of bacterial isolates, wherein the said method comprising the steps of:
a) isolating the genomic DNA from activated biomass;
b) amplifying the target DNA with commercially available appropriate primers using genomic DNA as a template obtained from step (a) to obtain random DNA sequences;
c) evaluating all the random DNA sequences obtained from step (b) using genomic DNA obtained from step (a) to get amplified primers;
d) selecting the primers obtained from step (c) based on the number and size of the bands wherein the larger number of bands indicates the high efficiency of the said primer;
e) checking the performance of selected primers obtained from step (d) using different concentration of magnesium ions and different annealing temperature;
f) evaluating the selected six primers obtained from step (d) using genomic DNA isolated from different bacteria present in the sludge sample for screening the bacterial isolates.

In an embodiment of the present invention, the activated biomass is selected from the group consisting of any waste including wastewater of any industry.

In another embodiment of the present invention, one of the industries selected from the group consisting of pesticide industry, dye industry, refinery, petrochemical industry, refinery wastewater, mixed pesticide and pharmaceutical waste etc.

Further, in another embodiment of the present invention, the genomic DNA is isolated by known methods.

In yet another embodiment of the present invention, the primers used for obtaining random DNA sequences comprising the following sequences:

a) RAPD primer 1: 5′ d[GGTGCGGGAA]3′
b) RAPD primer 2: 5′ d[GTTTCGCTCC]3′
c) RAPD primer 3: 5′ d[GTAGACCCGT]3′
d) RAPD primer 4: 5′ d[AAGAGCCCGT]3′
e) RAPD primer 5: 5′ d[AACGCGCAAC]3′
f) RAPD primer 6: 5′ d[CCCGTCAGCA]3′

In another embodiment of the present invention, the selected primers consisting of all or part of the following sequences:

a. SEQ ID NO 1: 5′-TTGATATCATGTCGACCTATCCAG-3′;
b. SEQ ID NO 2: 5′-TTCGTTCCGTCCTGCAGCCTCAAT-3′;
c. SEQ ID NO 3: 5′-GCAAGCTTGGCGATTACA-3′;
d. SEQ ID NO 4: 5′-TGCCAGGATATCAGACAGATG-3′;
e. SEQ ID NO 5: 5′-GGCCAACGCGGCC-3′;
f. SEQ ID NO 6: 5′-CCTGCAGCAGA-3′.

Further, in another embodiment of the present invention, the performance of selected primers is checked using magnesium ions concentration ranging from 1.5 mM to 3 mM. In yet another embodiment of the present invention, the performance of selected primers are checked at different annealing temperature in the range from 30 degree C. to 50 degree C.

Further, the present invention also provides a kit for screening of bacterial isolates, wherein the said kit comprising;
a) instructions for screening the bacterial isolates;
b) suitable reagents for performing PCR;
c) composition of six primers.

Present invention discloses a method to determine the differences in DNA samples based on the distance and occurrences of selected inverted repeat sequences. In particular, this invention relates to designing and evaluating the primers and amplification conditions to arrive at set of primers, which have application in differentiating DNA samples. The sample could have arrived from closely related strains from the same genera or environmental samples collected from different areas or from same place but different time points.

The main aim of the present invention is to design primers and amplification conditions, which can differentiate closely, related strains from the same genera, based on the genomic content. There are some commercially available RAPD primers as shown in Table 1. However, these primers have not been evaluated for environmental isolates. In environmental isolates, genotypic diversity is crucial irrespective of their taxonomic relationship or similarities (Harita et al. 2003, Kutty et al. 2000, Kutty et al. 2001; Moharikar et al. 2003). There are recent advances to address these issues of diversity. These have been reported for different scenarios of active biomass employed for waste management (Watanabe and Hino 1996; Widada et al. 2002; Yuan and Blackall 2002; Purohit et al. 2003).

TABLE 1
Standard Primers Used (Commercially available
from M/s Amersham Inc, UK.)

| Primer Name | Primer Sequence | Available from |
|---|---|---|
| RAPD primer 1 | 5′ d[GGTGCGGGAA]3′ | Amersham Pharmacia |
| RAPD primer 2 | 5′ d[GTTTCGCTCC]3′ | Amersham Pharmacia |
| RAPD primer 3 | 5′ d[GTAGACCCGT]3′ | Amersham Pharmacia |
| RAPD primer 4 | 5′ d[AAGAGCCCGT]3′ | Amersham Pharmacia |
| RAPD primer 5 | 5′ d[AACGCGCAAC]3′ | Amersham Pharmacia |
| RAPD primer 6 | 5′ d[CCCGTCAGCA]3′ | Amersham Pharmacia |

Moreover, inventors have developed various methodologies where specific bacteria could be targeted and have developed molecular monitoring tools (Kapley et al. 2000, Kapley et al. 2001). This invention provides a tool can be used for discrimination of pure cultures or time dependent perturbations realized by total microbial population. The invention describes the development of a method to study microbial diversity. It involves optimization of conditions in which the primer, which is represented in the DNA samples, as an inverted repeat is best exploited during the PCR. There are three types of Taq Polymerase preparations easily available in the market. Using one of the commercially available primers, the optimization was carried out. The primers designed in the lab were then compared for their performance in RAPD in defined reaction conditions.

Optimization and designing of methodology was carried out and the following components were used as described in Table 2.

TABLE 2
PCR reaction

| Components | Final Concentration |
|---|---|
| 10X PCR buffer* | 1 X |
| MgCl₂ solution | Variable (1 mM-5 mM) |
| 10 mM dNTP mix | 200 μm each dNTP |
| Primer | 25 pmol |
| Enzyme | 3 units |
| Template | 25-500 ng/reaction |
| Distilled water | To make final volume of 50 μl |
| Total volume of reaction | 50 μl |

*PCR buffer and enzymes tried:
1. Perkin Elmer, USA
2. Invitrogen Life Technologies, USA
3. Bangalore Genei, India

The following examples are given by way of illustration of the present invention and should not be construed to limit the scope of present invention.

### Example-1

### RAPD of Single Template with Different Commercially Available Enzymes

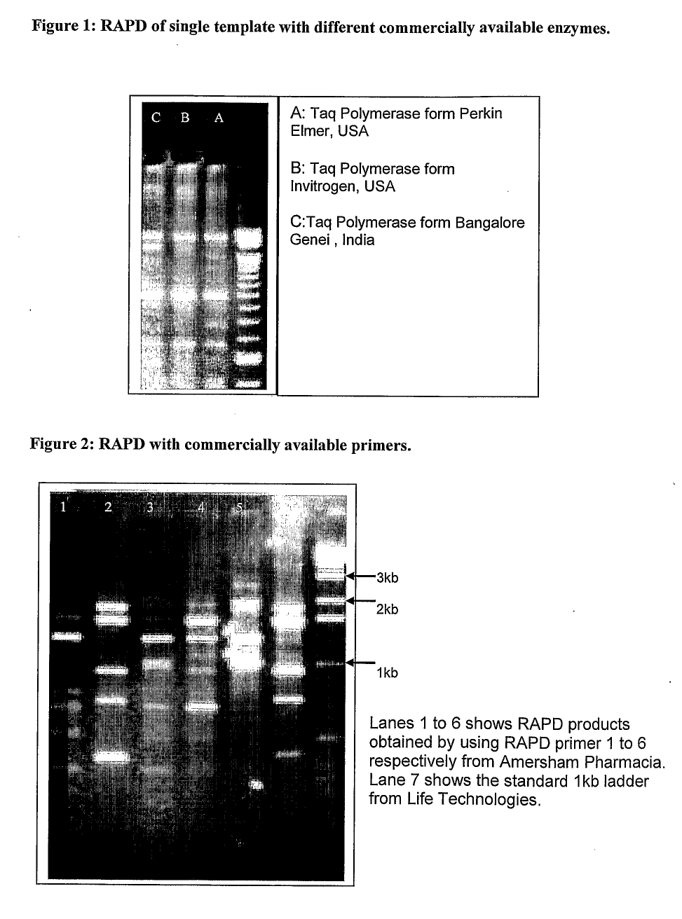

Initially, ˜25 ng total DNA from activated biomass was used as template with RAPD primer 5 from Amersham Pharmacia. Reaction components used were 10×PCR buffer from (a) Perkin Elmer, USA (b) Invitrogen Inc. USA and (c) Bangalore Genei, India with MgCl₂ concentration of 3 mM and 3 units of enzyme. The rest of the reaction conditions were used as per the manufacturer's instructions. All other components were used as described in Table 2. The thermocycling parameters used were 40 cycles of 94° C. for 1 min, 37° C. for 1 min and 72° C. for 1 min using Perkin Elmer Thermocycler Model 9600. After the completion of cycle, 10 μl of reaction was analyzed on 0.8% agarose gel electrophoresis as shown in FIG. 1. Lane No. C shows the presence of more bands with higher density. The experiments were carried out using different samples that gave reproducibility of results. Hence, for all the experiments through out the process of methodology development, Taq Polymerase from M's Bangalore Genei was used with their buffer system.

### Example-2

### RAPD with Commercially Available Primers

Reaction condition used were as follows—

| | |
|---|---|
| Template: | ~25 ng total DNA from activated biomass (1:3 diluted) |
| Primer: | RAPD primer 1-6 from Amersham Pharmacia. |
| Enzyme: | 3u Taq polymerase from Bangalore Genei. |
| MgCl₂: | 3 mM. |

All other components were used as described in Table 1.

Thermo cycling parameters: 40 cycles of 94° C. for 1 min, 37° C. for 1 min and 72° C. for 1 min and the gel picture is shown in FIG. 2. The primers 2, 5 and 6 show multiple bands. However, the aim was to generate a fingerprinting pattern of template DNA with many amplified DNA sequences; hence different MgCl₂ concentrations were tried to ensure possible flexibility demonstrated by selected primer in the reaction.

### Example-3

### Designing of RAPD Primers

The primers have been artificially designed and commercially synthesized. Random DNA sequences were generated using suffix tree algorithms to give sub-strings of various sizes between 10-20 mers. The generated sequences were numbered. Thirty-one such sequences were picked as shown in Table 3.

TABLE 3
RAPD primers used in this study

| S. No. | Primer Sequence (5′-3′) |
|---|---|
| Pr 1. | AAATGCATGCTTGGCGCTGATGGTGC |
| Pr 2. | CGGATGCATATGGATTGCATCACCGGC |
| Pr 3. | AAATAYCTGATYGGYGCYGAYGG |
| Pr 4. | GGCGGATGSYDRTGVVBBGCGTCGCC |
| Pr 5. | CGATCCGTAACTGGTCTGAG |
| Pr 6. | GCCGGTGCTTATTCTGTC |
| Pr 7. | TTGATATCATGTCGACCTATCCAG |
| Pr 8. | TCAACCGCGGCGCACAAGCATC |
| Pr 9. | TTCGTTCCGTCCTGCAGCCTCAAT |
| Pr 10. | GTITGGCACTCGAGGCCCGAIG |
| Pr 11. | GCAAGCTTGGCGATTACA |
| Pr 12. | TGCCAGGATATCAGACAGATG |
| Pr 13. | ACGTGAATTCCATGAACGACATGAACGCT |
| Pr 14. | AAGGCCTCTTACCCTTG |
| Pr 15. | ACGTGAATTCCCATGAGGGTAATAAATAATG |
| Pr 16. | CAATCCCGGGCCCTAGCGCGTAACTACC |
| Pr 17. | CAATCCCGGGTCCGGGGTCCCTCCTATTAAAAC |
| Pr 18. | AGCTGGATCCGTAAAAGTGGTCACTGGATCAC |
| Pr 19. | ACGTGAATTCATTGCGGCCGCATTGCA |
| Pr 20. | ACGTCATATGAATGCATGCAATGCGGC |
| Pr 21. | ACGTCATATGAATGCATG |
| Pr 22. | ACGTGGTACCATTGCCGCCGC |
| Pr 23 | GGCCAACGCGGCC |
| Pr 24. | CCTGCAGCAGA |

| | | |
|---|---|---|
| Pr 25. | C(A/C)CG(C/T)C(A/G)(C/G)CA | |
| 26. 1 | Amersham Kit | d[GGTGCGGGAA] |
| 27. 2 | Amersham Kit | d[GTTTCGCTCC] |
| 28. 3 | Amersham Kit | d[GTAGAGCCGT] |
| 29. 4 | Amersham Kit | d[AAGAGCCCGT] |
| 30. 5 | Amersham Kit | d[AACGCGCAAC] |
| 31. 6 | Amersham Kit | d[CCCGTCAGCA] |

Underlined sequences are the six final RAPD primers recommended by the inventor

### Example-4

### Evaluation of All Primers Shown in Table 3

A) Using DNA Isolated from Activated Sludge:

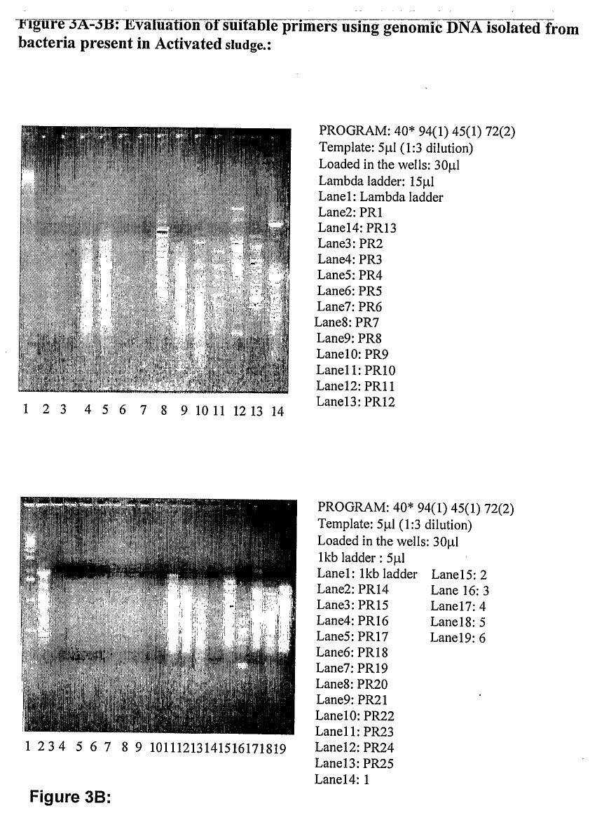

For evaluation, activated sludge sample has been used. This represents mixture of various kinds of genomic DNA derived from wide variety of bacterial species constituting an active population. The primers were evaluated using same experimental conditions as were used in Example 2. All the primers were commercially synthesized and column purified for its application in PCR reaction and the results obtained are shown in FIG. 3A-3B. Out of the designed 31 primers 17 primers gave amplification. Out of these 17 primers six primers were selected based on the number of bands and sizes of the bands when analyzed on 1.5% agarose gel.

B) Using DNA Isolated from Pure Cultures:

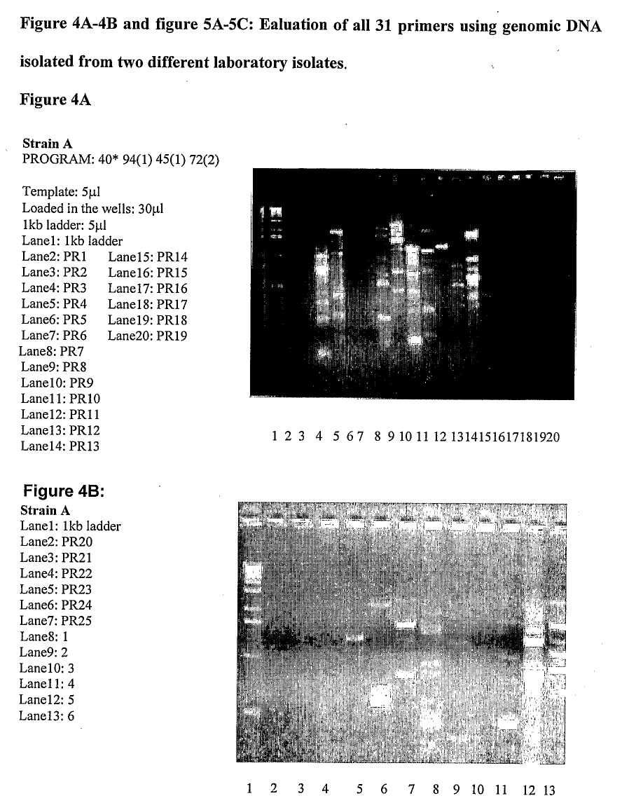

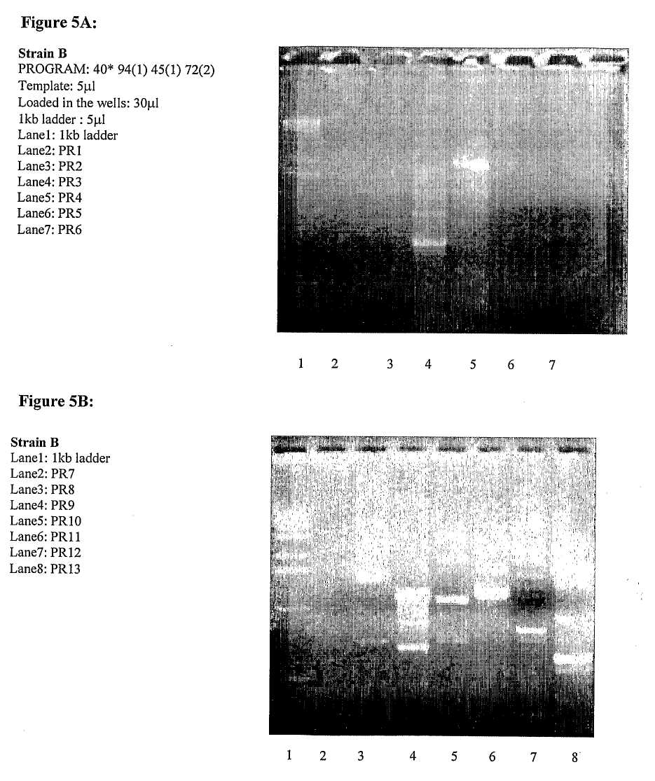

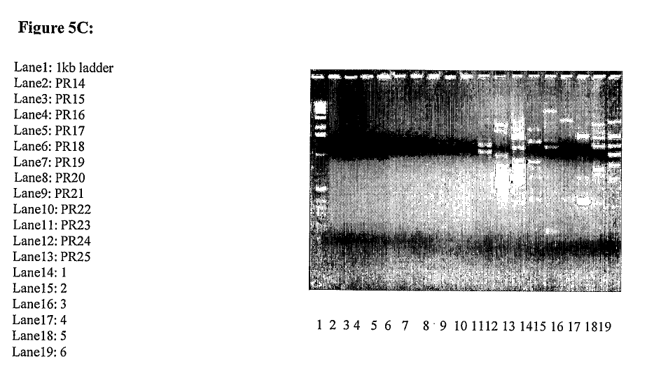

All 31 primers were evaluated using same experimental conditions as were used in Example 2. For the study, two laboratory isolates were used as shown in FIG. 4A-4B and FIG. 5A-5C.

The amplification patterns when compared using BioNumerics version 3.5 software indicated that there are six primers, which give different bands of various sizes. Similar results were also observed with activated sludge sample as shown in FIG. 3A-3B.

### Example-5

### Performance of Six Selected Primers at Varying Annealing Temperatures

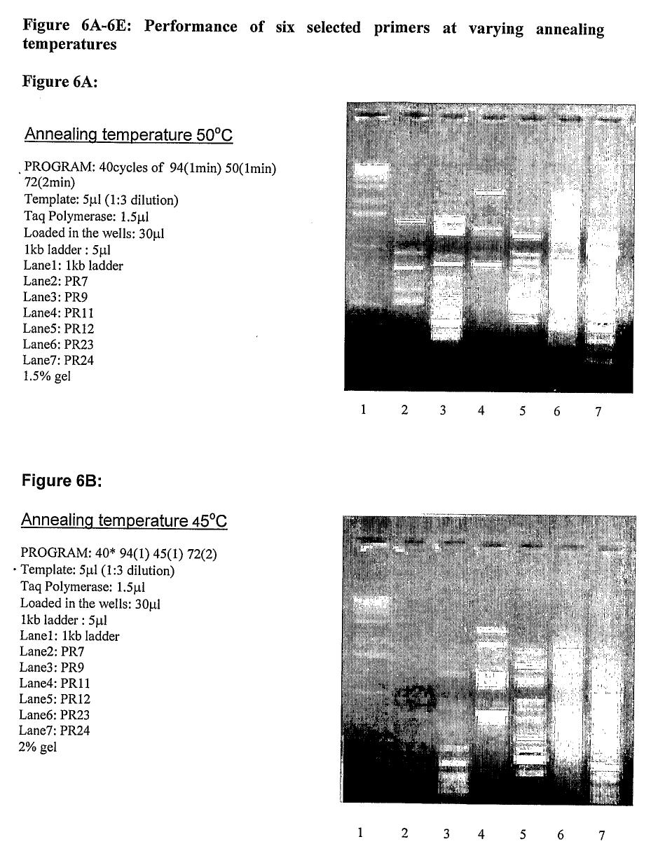

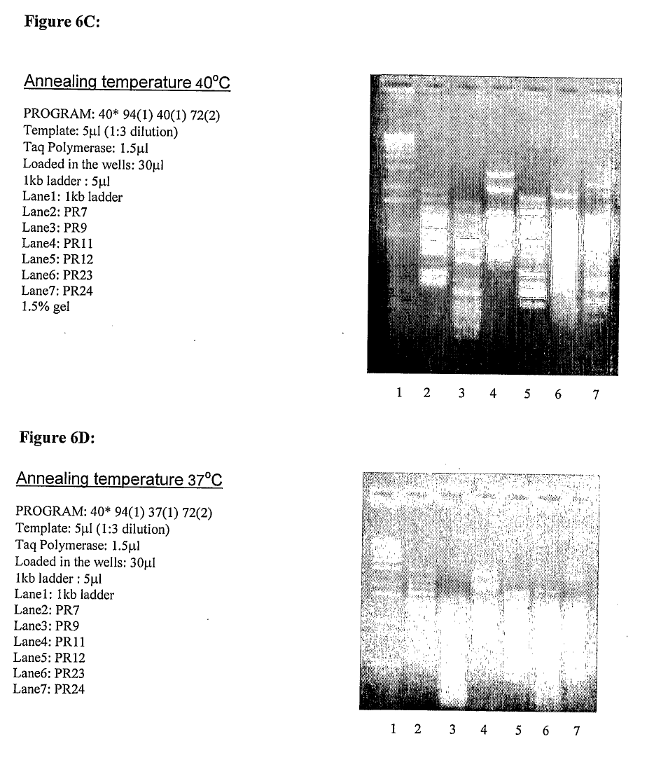

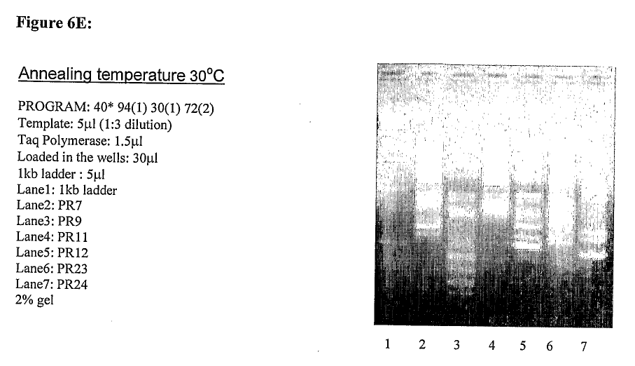

By varying the annealing temperature in PCR reaction, the experiments were carried out using amplification conditions as described in Example 2 and are shown in FIG. 6A-6E. All the primers yielded better amplification pattern than the commercially available primers. However to make sure the credibility of the selected six primers, the annealing temperature, which decides the criterion for fetching the inverted repeat has been evaluated in this example.

### Example-6

### Performance of Six Primers and its Relation to Magnesium Ion Concentration

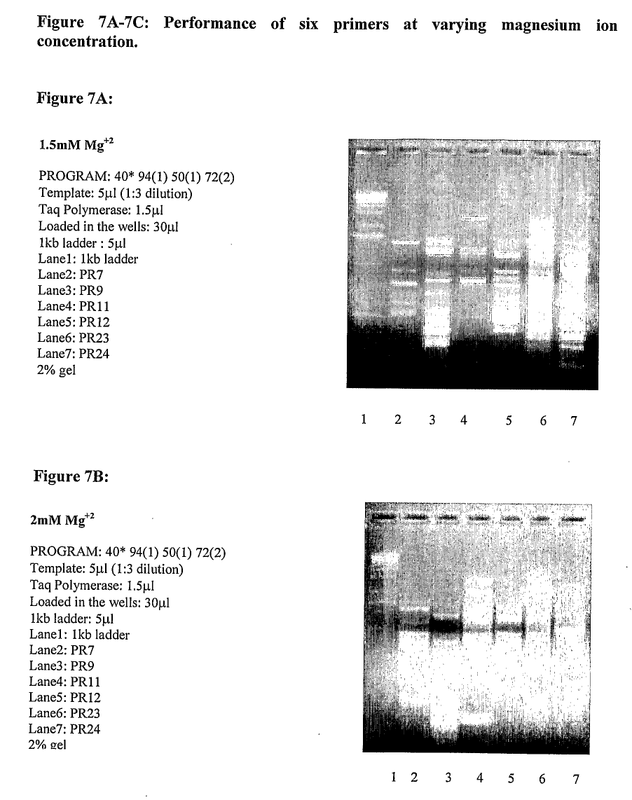

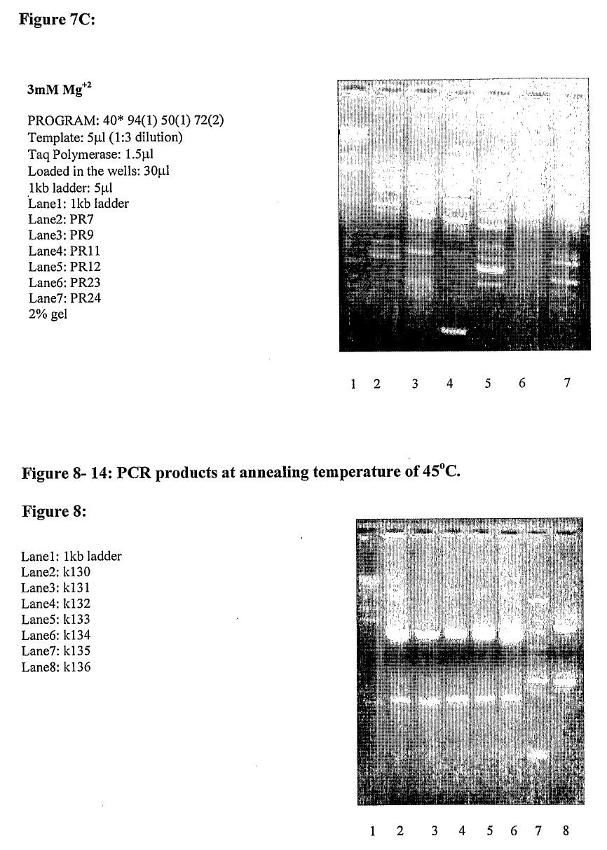

Varying the magnesium ion concentration in PCR reaction, the experiments were carried out using amplification conditions as described in Example 2 and shown in FIG. 7A-7C. The magnesium ion concentration decides the performance of enzyme used in this polymerization reaction specifically with reference to final size of the polymerization product.

### Example-7

### Evaluation of Six Selected Primers Using DNA Isolated from Various Bacteria

The primers were tested with different bacterial isolates from different activated sludge sample utilizing different phenolics and chlorinated residues as substrates. The details of sludge and their origin are provided in Table 4.

TABLE 4
Sludge S1 to S8 obtained from ETP treating
wastewater from following industries

| S. No. | Activated sludge collected |
|---|---|
| S1 | Pesticide industry |
| S2 | Dye industry |
| S3 | Pesticide industry |
| S4 | Refinery |
| S5 | Petrochemical Industry |
| S6 | Refinery wastewater |
| S7 | Refinery wastewater |
| S8 | Mixed pesticide and pharmaceutical waste |

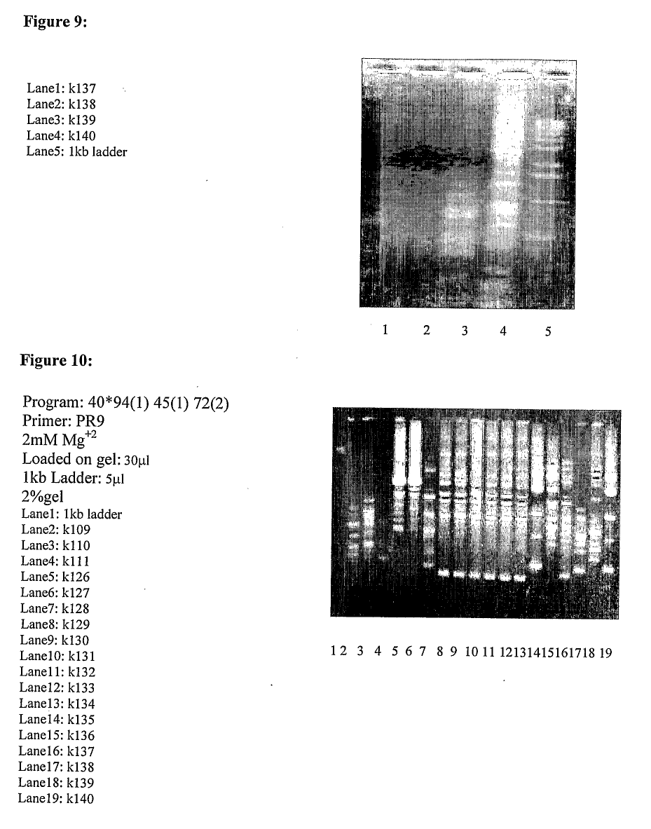

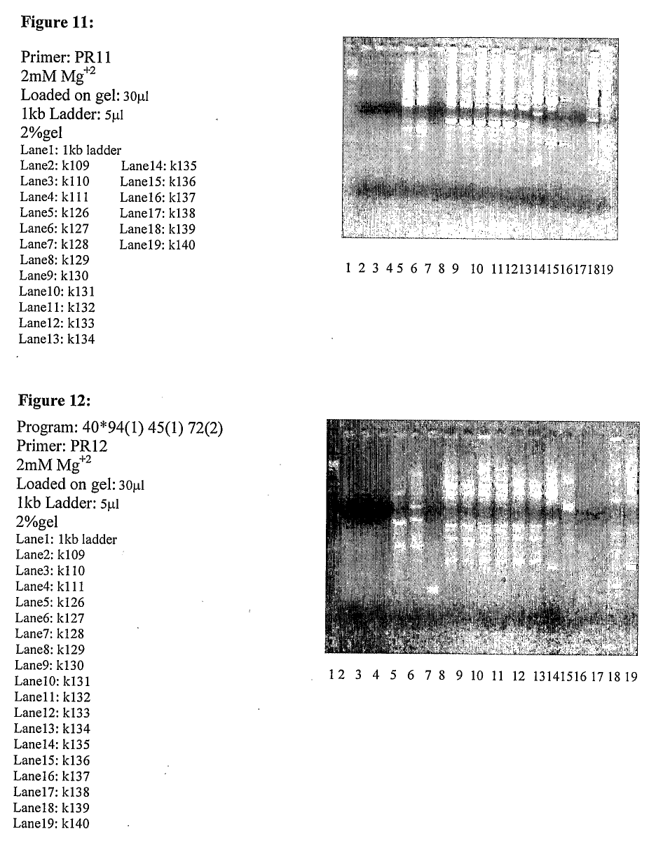

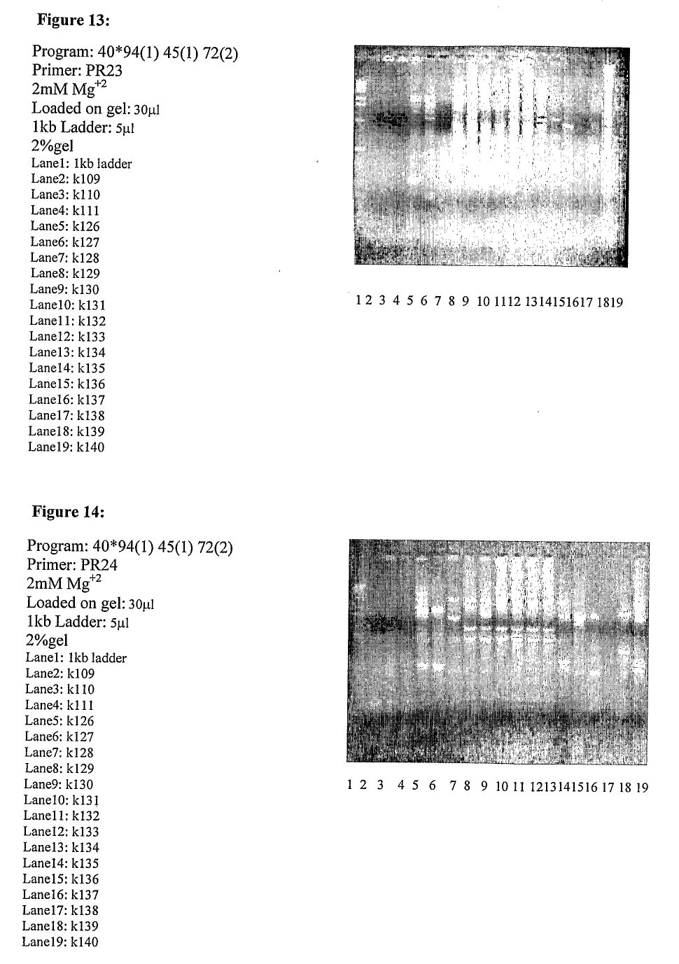

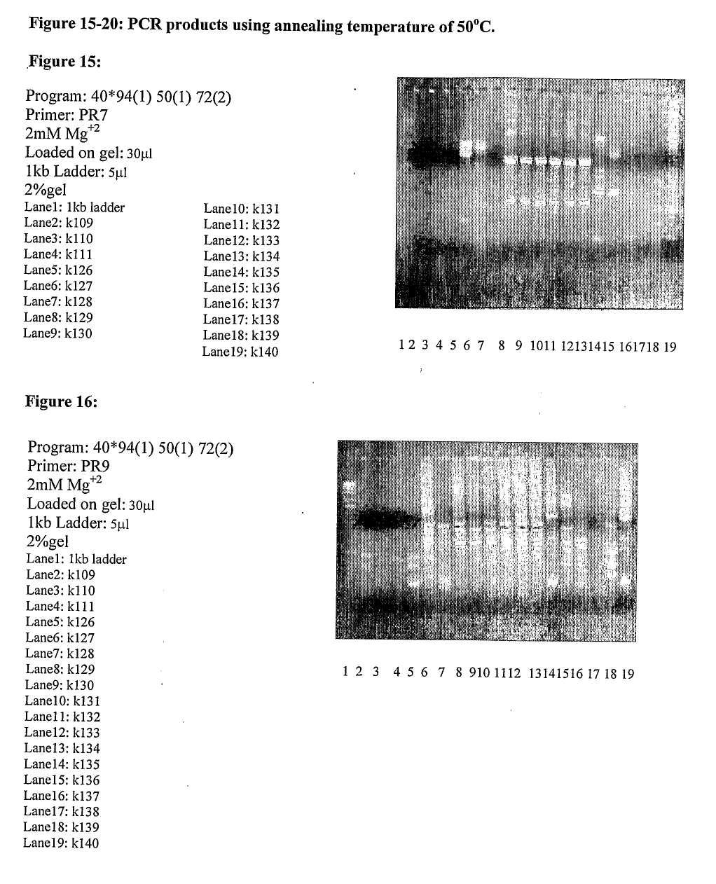

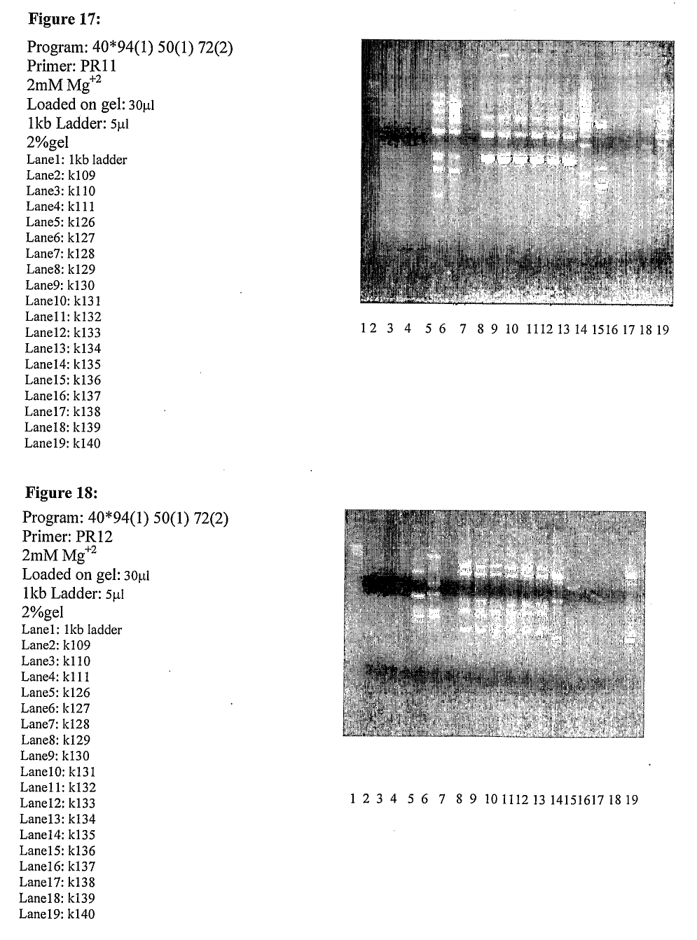

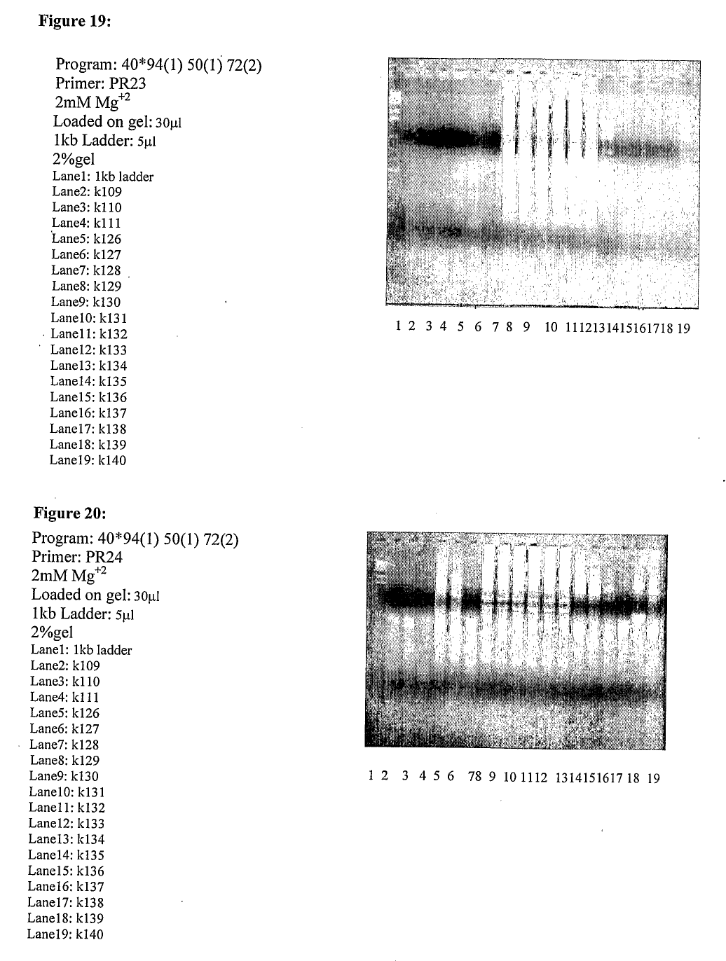

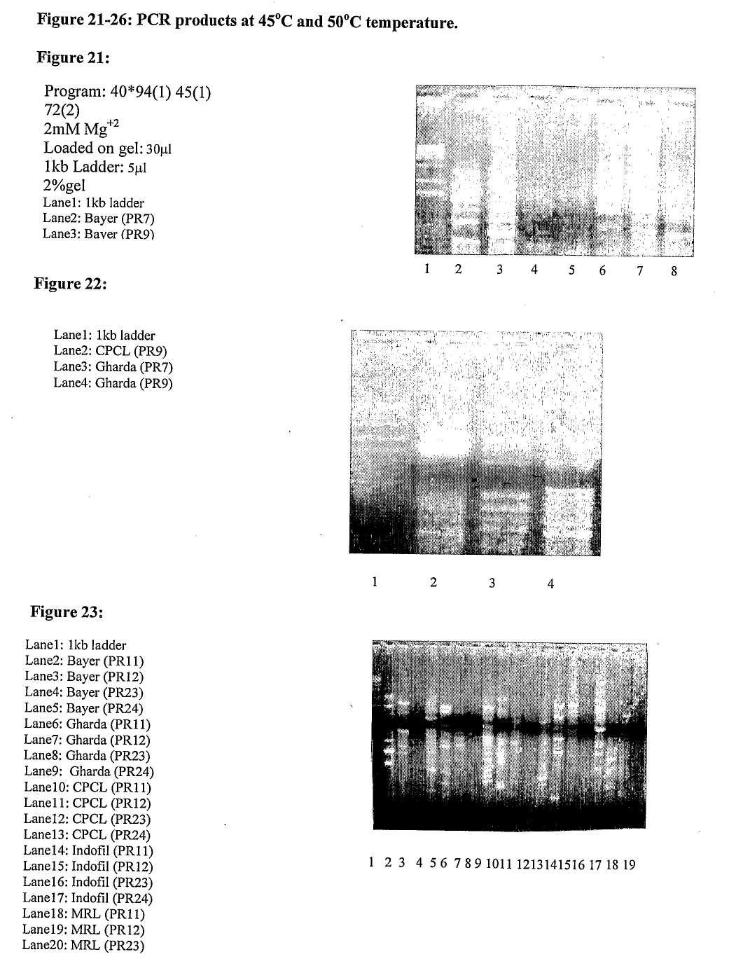

The isolates K109 to K140 were used in this example. The total DNA for each isolate has been prepared and the same amount of DNA was used with all the six primers in the optimized conditions arrived from Example 4-6. FIG. 8-15 shows the results were annealing temperature of 45° C. was used for 1 min in PCR reactions, whereas FIG. 16-21 shows the results using annealing temperature of 50° C. for 1 min in PCR reactions. Results show that annealing temperature of 45° C. was preferred to demonstrate the diversity of bacterial isolates.

### Example-8

### Demonstration of Six Selected Primers Using DNA Isolated from Different Sludge Samples to Show Associated Genetic Diversity

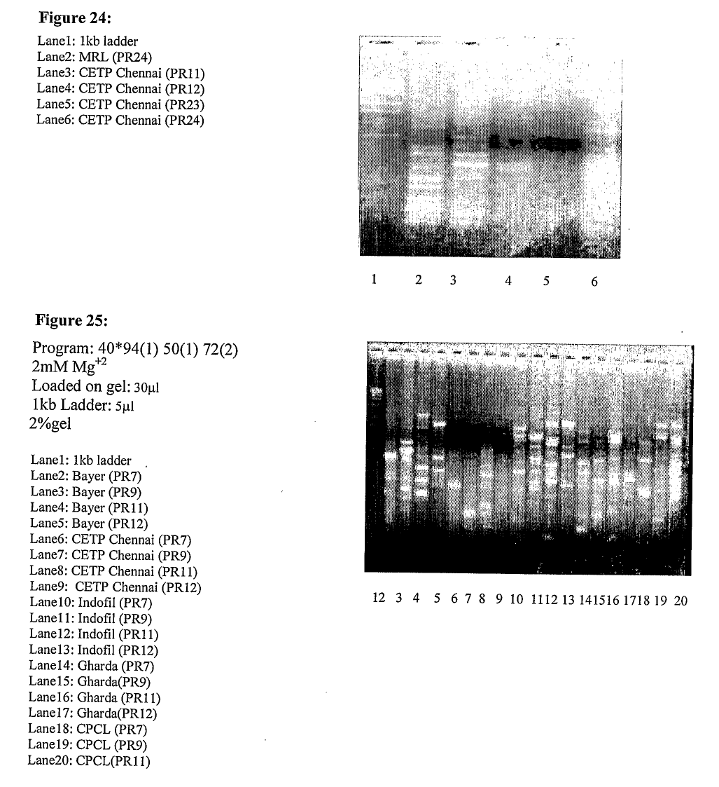

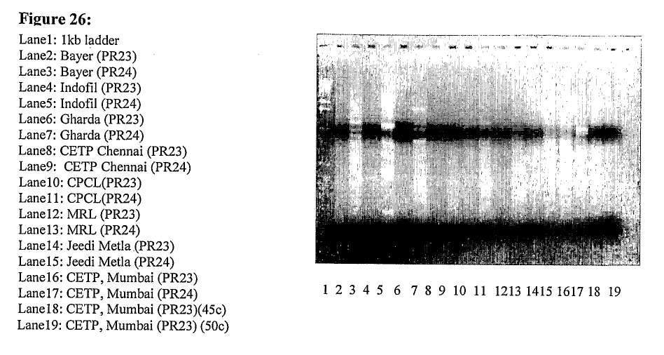

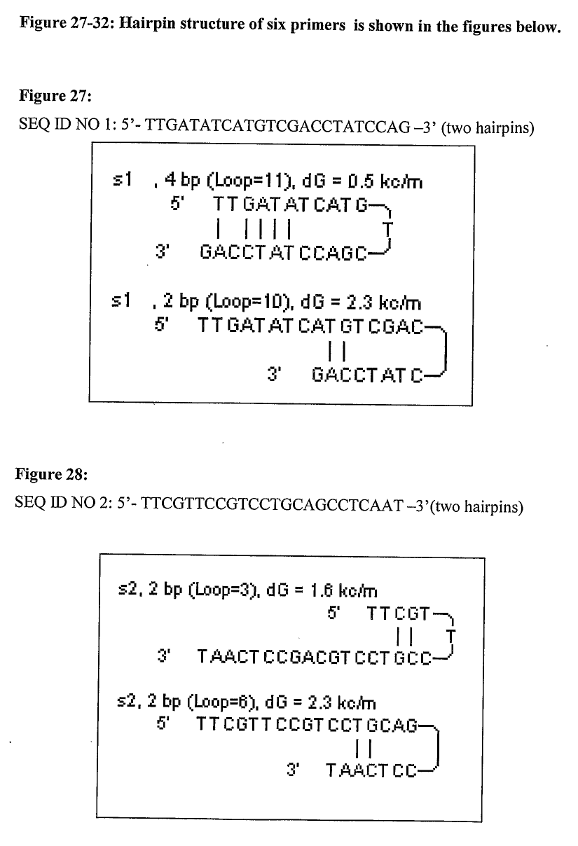

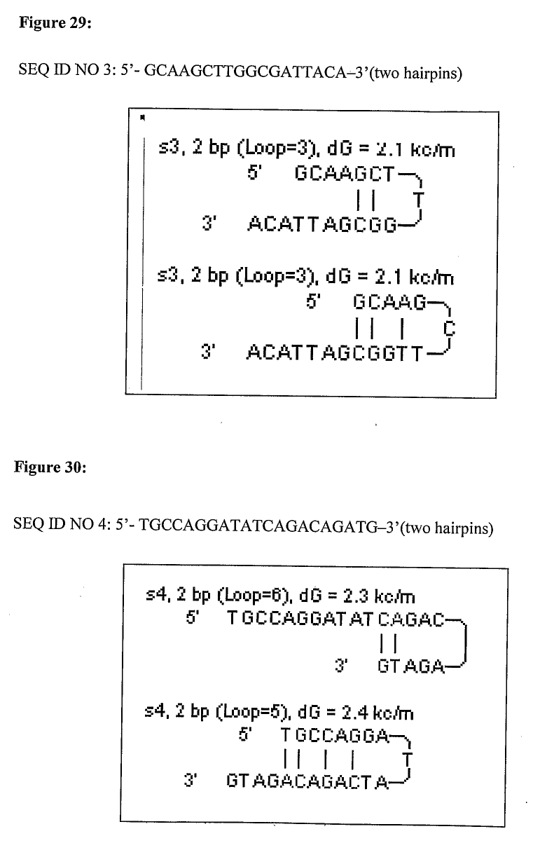

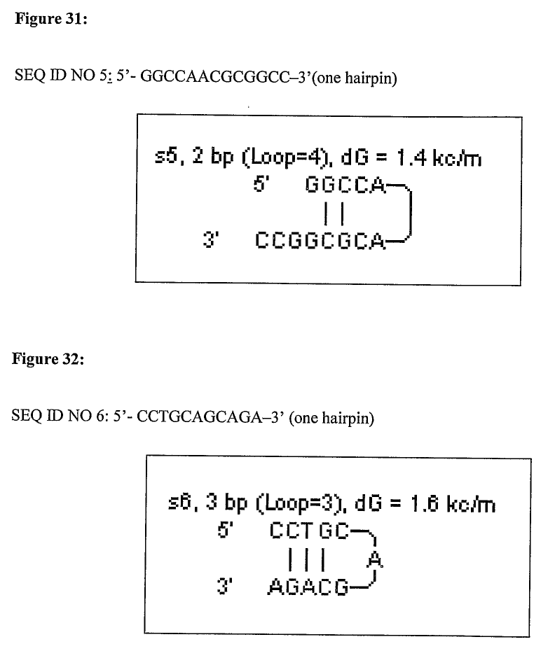

The total DNA was prepared from the sludge samples described in Table 4. The DNA sample was diluted with sterilized distilled water to give 25 ng/μl concentration and was used in this example. The associated DNA diversity of the sample has been reflected in FIG. 22-27 where 45° C. and 50° C. temperatures were used for 1 min in PCR reactions. The results corroborated with those obtained in Example 7 for different isolates. In conclusion, the invented method defines six selected primers as shown in Table 3 that can be used with annealing temperature between 45° C. to 50° C. for differentiating bacterial isolates and/or total microbial diversity of a defined niche.

### Advantages:

The main advantages of the present invention are:
The present invention provides a rapid method to distinguish bacterial genotypes and identify perturbations related to stress in any defined environmental niche unlike the methods described above in prior art.

We claim:

1. A composition comprising six primers useful for screening of bacterial isolates, wherein the primer sequences consisting of all or part of the following sequence ID;
a) SEQ ID NO 1: 5′-TTGATATCATGTCGACCTATCCAG-3′;
a) SEQ ID NO 2: 5′-TTCGTTCCGTCCTGCAGCCTCAAT-3′;
b) SEQ ID NO 3: 5′-GCAAGCTTGGCGATTACA-3′;
c) SEQ ID NO 4: 5′-TGCCAGGATATCAGACAGATG-3′;
d) SEQ ID NO 5: 5′-GGCCAACGCGGCC-3′;
e) SEQ ID NO 6: 5′-CCTGCAGCAGA-3′.

2. A composition as claimed in claim 1, wherein the said individual primers have the following characteristics:
a) SEQ ID NO 1 have the following characteristics:
(i) melting temperature (degree C.)=52.6 degree C.;
(ii) number of hairpin loops <2;
(iii) number of dimers <2;
(iv) number of internal loops=2;
(v) molecular weight=7920 Da;
b) SEQ ID NO 2 have the following characteristics:
(i) melting temperature (degree C.)=64.5 degree C.;
(ii) number of hairpin loops <2;
(iii) number of dimers <2;
(iv) number of internal loops=2;
(v) molecular weight=7920 Da;
c) SEQ ID NO 3 have the following characteristics:
(i) melting temperature (degree C.)=49.6 degree C.;
(ii) number of hairpin loops <2;
(iii) number of dimers <2;
(iv) number of internal loops=2;
(v) molecular weight=5940 Da;
d) SEQ ID NO 4 have the following characteristics:
(i) melting temperature (degree C.)=49.9 degree C.;
(ii) number of hairpin loops <2;
(iii) number of dimers <2;
(iv) number of internal loops=2;
(v) molecular weight=6930 Da
e) SEQ ID NO 5 have the following characteristics:
(i) melting temperature (degree C.)=51.5 degree C.;
(ii) number of hairpin loops <2;
(iii) number of dimers <2;
(iv) number of internal loops=2;
(v) molecular weight=4290 Da
f) SEQ ID NO 6 have the following characteristics:
(i) melting temperature (degree C.)=59.5 degree C.;
(ii) number of hairpin loops <2;
(iii) number of dimers <2;
(iv) number of internal loops=2;
(v) molecular weight=3630 Da

3. A composition as claimed in claim 1, wherein the said primers specifically hybridize to DNA inverted repeats and uniquely maps different sites in total DNA of an environmental origin or eubacteria.

4. A composition as claimed in claim 1, wherein the said primers are used for RAPD analysis for assessment of diversity of genera, different isolates or total DNA of any environmental or any origin

5. A method for screening of bacterial isolates, wherein the said method comprising the steps of:
a) isolating the genomic DNA from activated biomass;
b) amplifying the target DNA with commercially available appropriate primers using genomic DNA as a template obtained from step (a) to obtain random DNA sequences;
c) evaluating all the random DNA sequences obtained from step (b) using genomic DNA obtained from step (a) to get amplified primers;
d) selecting the primers obtained from step (c) based on the number and size of the bands wherein the larger number of bands indicates the high efficiency of the said primer;
e) checking the performance of selected primers obtained from step (d) using different concentration of magnesium ions and different annealing temperature;
f) evaluating the selected six primers obtained from step (d) using genomic DNA isolated from different bacteria present in the sludge sample for screening the bacterial isolates.

6. A method as claimed in claim 5, wherein the activated biomass is selected from the group consisting of any waste including wastewater of any industry.

7. A method as claimed in claim 6, wherein one of the industry selected from the group consisting of pesticide industry, dye industry, refinery, petrochemical industry, refinery wastewater, mixed pesticide and pharmaceutical waste etc.

8. A method as claimed in claim 5, wherein genomic DNA is isolated by known methods.

9. A method as claimed in claim 5, wherein the primers used for obtaining random DNA sequences comprising the following sequences:
a) RAPD primer 1: 5′ d[GGTGCGGGAA]3′
b) RAPD primer 2: 5′ d[GTTTCGCTCC]3′
c) RAPD primer 3: 5′ d[GTAGACCCGT]3′
d) RAPD primer 4: 5′ d[AAGAGCCCGT]3′
e) RAPD primer 5: 5′ d[AACGCGCAAC]3′
f) RAPD primer 6: 5′ d[CCCGTCAGCA]3′

10. A method as claimed in claim 5, wherein the selected primers consisting of all or part of the following sequences:
a. SEQ ID NO 1: 5′-TTGATATCATGTCGACCTATCCAG-3′;
b. SEQ ID NO 2: 5′-TTCGTTCCGTCCTGCAGCCTCAAT-3′;
c. SEQ ID NO 3: 5′-GCAAGCTTGGCGATTACA-3′;
d. SEQ ID NO 4: 5′-TGCCAGGATATCAGACAGATG-3′;
e. SEQ ID NO 5: 5′-GGCCAACGCGGCC-3′;
f. SEQ ID NO 6: 5′-CCTGCAGCAGA-3′.

11. A method as claimed in claim 5, wherein the performance of selected primers is checked using magnesium ions concentration ranging from 1.5 mM to 3 mM.

12. A method as claimed in claim 5, wherein the performance of selected primers are checked at different annealing temperature ranging between 30 degree C. to 50 degree C.

13. A kit for screening of bacterial isolates, wherein the said kit comprising;
a) instructions for screening the bacterial isolates;
b) suitable reagents for performing PCR;
c) composition of six primers as claimed in claim 1.

14. A composition, a method for screening of bacterial isolates and a kit thereof substantially as herein described with reference to the examples and drawings accompanying this specification.
